(19) United States
(12) Patent Application Publication
(10) Pub. No.: US 2010/0210940 A1
(43) Pub. Date: Aug. 19, 2010

(54) CT-Guided Focused Ultrasound for Stroke Treatment

(75) Inventors: William G. Bradley, La Jolla, CA (US); Thilo Hoelscher, San Diego, CA (US); Michael Brant-Zawadzki, Newport Beach, CA (US)

Correspondence Address: FISH & RICHARDSON, PC, P.O. BOX 1022, MINNEAPOLIS, MN, 55440-1022, US

(73) Assignees: HOAG MEMORIAL HOSPITAL PRESBYTERIAN, Newport Beach, CA (US); THE REGENTS OF THE UNIVERSITY OF CALIFORNIA, Oakland, CA (US)

(21) Appl. No.: 12/619,567

(22) Filed: Nov. 16, 2009

Related U.S. Application Data
(60) Provisional application No. 61/114,940, filed on Nov. 14, 2008.

(51) Int. Cl. A61B 5/05; A61N 7/00

(52) U.S. Cl. 600/427; 601/2

(57) ABSTRACT

Techniques and systems are disclosed for performing mechanical ablation of blood clots using low-intensity ultrasound. A system includes a computed tomography (CT) scanner to acquire axial scans of a target subject containing at least one intravascular blood clot, and generate volumetric CT data of the target subject based on the acquired axial scans. A data processing device is in communication with the CT scanner to process the generated volumetric CT data, and identify at least one parameter of an ultrasound beam to ablate the at least one intravascular blood clot based on the processed volumetric CT data. Also, an ultrasound ablation device is in communication with the data processing device to generate the ultrasound beam based on the identified at least one parameter, and apply the generated ultrasound beam with the identified at least one parameter to ablate the at least one intravascular blood clot.

### CLAIM OF PRIORITY

This application claims priority under 35 USC §119(e) to U.S. Patent Application Ser. No. 61/114,940, filed on Nov. 14, 2008, the entire contents of which are hereby incorporated by reference.

### BACKGROUND

This application relates to devices and techniques that use ultrasound beams to ablate blood clots for stroke treatment, for example.

Magnetic resonance (MR)-guided focused ultrasound can be used to thermally disrupt intravascular thrombi in the setting of acute stroke. When an ultrasound beam pass through tissues, some of the beam is absorbed and converted to heat. Focused ultrasound beams can be applied deep in tissues to use the generated heat to thermally ablate a target blood clot, for example. However, high powered focused ultrasound beams can cause ultrasound induced blood-brain barrier breakdown. Also, the increased temperature can also cause unpredictable tissue damages. Moreover, MR has technical challenges in localizing a clot within the vascular space of an acutely ill patient who may need continuous monitoring and support.

### SUMMARY

Techniques, systems and apparatus are disclosed for implementing mechanical ablation of clots in brain for stroke treatment, for example.

In one aspect, a system includes a computed tomography (CT) scanner to acquire axial scans of a target subject containing at least one intravascular blood clot, and generate volumetric CT data of the target subject based on the acquired axial scans. A data processing device is in communication with the CT scanner to process the generated volumetric CT data, and identify at least one parameter of an ultrasound beam to ablate the at least one intravascular blood clot based on the processed volumetric CT data. An ultrasound ablation device is in communication with the data processing device to generate the ultrasound beam based on the identified at least one parameter, and apply the generated ultrasound beam with the identified at least one parameter to the target subject to ablate the at least one intravascular blood clot.

Implementations can optionally include one or more of the following features. The at least one parameter of the ultrasound beam can include at least one of a transmit frequency, a peak intensity, a pulse length, an ultrasound duration, or a duty cycle of the ultrasound beam. The peak intensity can include a level that ablates the intravascular blood clot without causing heat induced tissue damage. The data processing device can be configured to process the generated volumetric CT data to obtain thickness or density information for a portion of the target subject using the obtained volumetric CT data. The data processing device can be configured to identify the at least one parameter of the ultrasound beam based on the obtained thickness or density information. The thickness information can include bone thickness of a skull. The data processing device can be configured to identify a correlation between the peak intensity of the ultrasound beam and the thickness of the skull by comparing a bone density and a bone thickness of the skull with acoustic signal absorption in the skull. The data processing device can generate a coefficient to compensate for inter-individual differences in bone density or bone thickness; and identify the at least one parameter of the ultrasound beam based on the generated coefficient.

In another aspect, a method included acquiring, at a computed tomography scanner, axial scans of a target subject containing at least one intravascular blood clot. At the computed tomography scanner, volumetric CT data of the target subject is generated based on the acquired axial scans. At a data processing device, the generated volumetric CT data is processed. At the data processing device, at least one parameter of an ultrasound beam is identified to ablate the at least one intravascular blood clot based on the processed volumetric CT data. At an ultrasound ablation device, the ultrasound beam is generated based on the identified at least one parameter. From the ultrasound ablation device, the generated ultrasound beam is applied with the identified at least one parameter to the target subject to ablate the at least one intravascular blood clot.

Implementations can optionally include one or more of the following features. The at least one parameter of the ultrasound beam can include at least one of a transmit frequency, a peak intensity, a pulse length, an ultrasound duration, or a duty cycle of the ultrasound beam. The peak intensity can include a level that mechanically ablates the at least one intravascular blood clot without heating tissues surrounding the intravascular blood clot. The applied ultrasound beam can be focused to the at least one intravascular blood clot. Processing the generated volumetric CT data can include obtaining thickness or density information for a portion of the target subject using the obtained volumetric CT data. The at least one parameter of the ultrasound beam can be identified based on the obtained thickness or density information. The thickness information can include bone thickness of a skull. A correlation between the peak intensity of the ultrasound beam and the thickness of the skull can be identified by comparing a bone density and a bone thickness of the skull with acoustic signal absorption in the skull. A coefficient can be generated to compensate for inter-individual differences in bone density or bone thickness. The at least one parameter of the ultrasound beam can be identified based on the generated coefficient.

In another aspect, the described techniques and, apparatus and system can be implemented as a computer readable medium embodying instructions that when executed by a processor cause a data processing apparatus to perform the described techniques.

The subject matter described in this specification potentially can provide one or more of the following advantages. By using low power ultrasound, ultrasound induced blood-brain barrier breakdown can be avoided. Also, the low powered ultrasound beam application does not generate high heat and thus no thermal effects are caused. In addition, CT can provide faster results than magnetic resonance imaging (MRI) in a more forgiving clinical environment. Further, CT is the preferred imaging technique to assess bone thickness

### BRIEF DESCRIPTION OF THE DRAWINGS

FIG. 1 shows an example system for implementing mechanical ablation of a target tissue.

FIG. 2 shows an example 3D patch sample distribution on the surface of the virtual skull based on the reconstructed CT data.

FIG. 3 shows the overall skull thickness distribution of all patches and the calculated mean thickness for the entire sample.

FIG. 4 shows an example mounting of a skull on an acrylic frame which is fixed to a plastic cover including a center hole to provide access to the skull cavity.

FIG. 5 shows an example high intensity focused ultrasound (HiFU) needle hydrophone.

FIG. 6 shows an example process for performing mechanical ablation using low-powered focused ultrasound.

FIG. 7 shows an example process for performing mechanical ablation using low-powered focused ultrasound to detect stroke.

Like reference symbols and designations in the various drawings indicate like elements.

### DETAILED DESCRIPTION

The techniques and systems described in this application can be used to perform mechanical ablation of blood clots in arteries supplying the brain. A low powered ultrasound beam can be used to mechanically ablate a blood clot in the brain without generating undesired heat. To identify target blood clots, computed tomography angiography (CTA) can be used to provide locations of intravascular blood clots.

Thermal Ablation

An ultrasound ablation system can be used to focus ultrasound beams on a target tissue to ablate the target tissue using heat generated by absorbed ultrasound waves. Thermal ablation of blood clots can be performed by varying or adjusting the power settings for the ultrasound ablation system based on various factors, such as the location of the clot and medical condition of the patient. To perform thermal ablation of clots in the brain, for example, acoustic power settings for the ultrasound ablation system can be varied between 500 Watts (in patients with thalamic pain, for example) and 2000 Watts (in patients with brain tumors, such as glioblastoma). For transcranial sonothrombolysis for example, power settings of 270 Watts (43% average clot weight loss) and 27 Watts (23% average clot weight loss) can be used.

Because such high powered focused ultrasound beams can generate significant levels of heat, an MR-based imaging system is needed to monitor the generated heat level. In addition, the generated heat can cause unpredictable tissue damage.

Mechanical Ablation System Using Low Power Focused Ultrasound

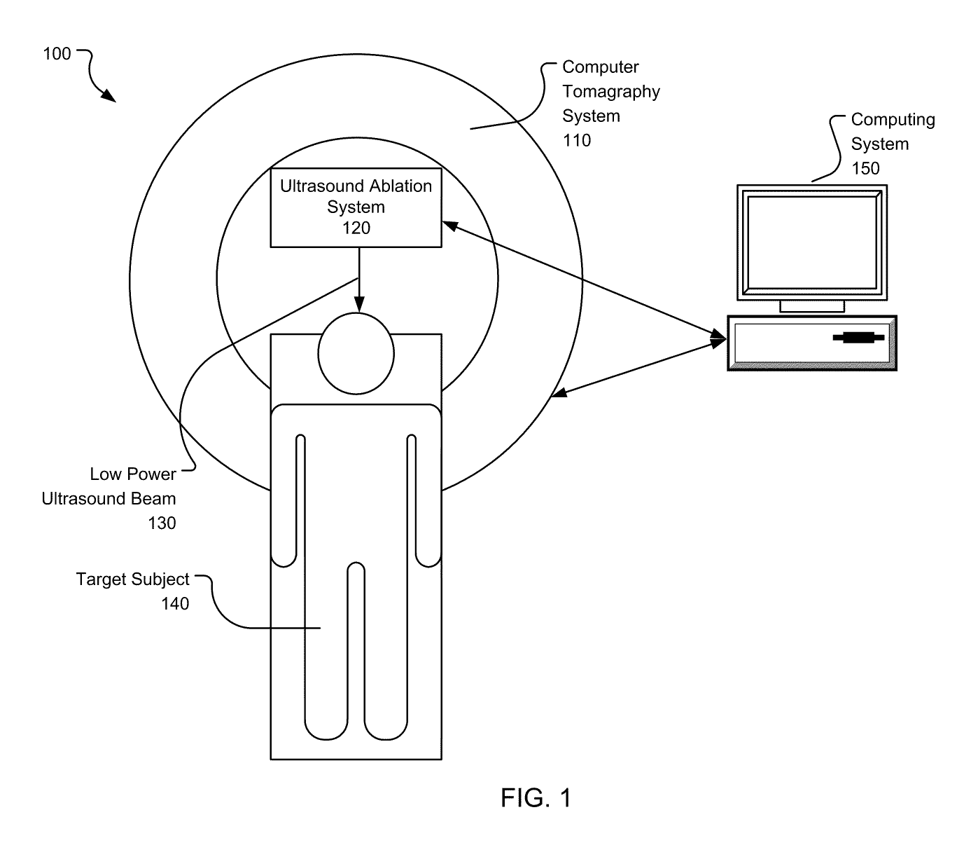

FIG. 1 shows an example system 100 for implementing mechanical ablation of a target tissue. The system 100 includes a CT system 110 and an ultrasound ablation system 120. The system 100 can optionally include a computing system 150 for controlling and operating the CT system 110 and the ultrasound ablation system 120. For example, the computing system 150 can receive CT data recorded by the CT system 110 and process the CT data to identify the location of a target tissue, such as a blood clot in an artery supplying blood to the brain.

The CT system 110 can include any one of the available commercial CT devices. For example, a 64-slice GE Discovery HD-750 CT scanner can be used to generate volume CT data sets of a skull of a target subject 140. High resolution CT scans of the skull can be acquired with a slice thickness of 0.23 mm and zero spacing between the slices, for example. The slices can be reconstructed using a bone filter.

An example ultrasound ablation system 120 includes the ExAblate™ 4000 High-Intensity Focused Ultrasound System (InSightec Inc., Tirat Carmel/Israel) that is implemented for brain applications. The ultrasound ablation system 120 includes a hemispheric phased array transducer with 1,000 single piezo elements that can be operated independently. Due to the geometry of the transducer, a sharp focus (2.0 mm in X/Y- and +/−3.0 mm in Z-orientation, for example) can be generated in the center of the transducer.

The ultrasound ablation system 120 can be operated at a low power range to use a low-powered focused ultrasound beam to perform mechanical ablation of clots without generating high heat. Such mechanical ablation process at low power ranges can be implemented to provide various advantages over thermal ablation process. For example, heat induced tissue damage can be avoided.

The ultrasound ablation system 120, such as the Insightec Exablate 4000 ultrasound transducer array, can be mated to a CT scanner rather than to an MRI scanner. While the Insightec ExAblate 4000 was primarily designed to thermally ablate lesions inside the brain under MR-guidance, the system 100 described in this specification can implement such ultrasound ablation system to operate in conjunction with a CT scanner rather than an MRI scanner. The Insightec ExAblate 4000 includes a hemisphere approximately 14 inches in diameter and 10 inches long with 1000 ultrasound transducers being focused on a point approximately 4 mm in diameter in XY orientation and 6 mm in diameter in Z orientation. The Insightec ExAblate 4000 can be mounted inside the bore of an MR scanner which serves to both focus the point of beam confluence and to measure the temperature rise. The Insightec ExAblate 4000 is described as an example system only and other similar focused ultrasound ablation systems can also be implemented.

The ultrasound ablation system 120 can be similarly mounted inside the bore of a CT scanner and use the CT angiogram to show the location of the intravascular thrombus to target the ultrasound beams. Because the ultrasound ablation system 120 is operated in low power mode to mechanically disrupt the thrombus, there is no need to measure or keep track of the temperature change.

Various other focused ultrasound is usually performed under MR guidance because MRI can be made sensitive to temperature change to ensure that thermal ablation has taken place and to navigate the focus towards the target. For example, focused ultrasound can be applied to thermally ablate uterine fibroids and painful boney metastases. To determine that the temperature has risen to at least 60° C. needed to cause coagulation necrosis, a technique called MR thermometry is used to monitor the temperature increase by monitoring a phase shift. Thus, MR guidance is needed in these other focused ultrasound ablation techniques to determine the amount of heat to deliver from a focused ultrasound unit.

In the stroke application described in this specification, the focused ultrasound ablation system 120 is operated in low power mode to mechanically disrupt a target clot instead of thermally ablating it. Therefore, an imaging modality can be used to identify the clot without needing to monitor the generated heat. CT angiography can be used as the imaging modality to identify the clot because it is not necessary to identify the temperature rise.

Under the low power mode, the focused ultrasound ablation system 120 can be operated at low power, such as at 10%, or less, of maximum power unless the skull is particularly thick, for example. The maximum power for the ExAblate 4000 may be up to 5000 Watts. The low power mode can include an example power range that includes 250, 500, and 750 watts with increasing skull thickness.

Focused Ultrasound Parameter Settings

An optimal parameter setting for performing mechanical ablation can be obtained by adjusting the power, duration of insonation, duty cycle, pulse length, etc. The following describes an example standard parameter setting that can be optimized:

Ultrasound modality: pulsed wave

Transmit frequency: 220 kHz

Peak Intensity: 200 W

Pulse length: 100 ms

Ultrasound duration: 30 s

Duty cycle: 50%

For example, based on data on extracranial sonothrombolysis using focused ultrasound by Rosenschein et al., Ultrasound Imaging-Guided Noninvasive Ultrasound Thrombolysis: Preclinical Results, Circulation (2000), 102:238-245 and Schafer et al., Influence OF Ultrasound Operating Parameters On Ultrasound-Induced Thrombolysis In Vitro, Ultrasound in Med. & Biol., Vol. 31, No. 6, pp. 841-847 (2005), a transcranial focused ultrasound setting can be optimized by testing at least four different parameters. For example, the effect of peak intensity (transmitted) and duration of insonation can be analyzed using combinations of different transmitted peak intensities and different durations of insonation. For example, combinations of ten different transmitted peak intensities (50 W-500 W, by increments of 50 W, for example) and five different durations of insonation (2 s, 10 s, 30 s, 60 s, and 120 s, for example) can be used to analyze the effect of peak intensity and duration of insonation. Multiple (e.g., at least ten) experiments can be performed for each set of parameters.

Also, the effect of pulse length and duty cycle can be analyzed. Once the optimal peak intensity/ultrasound duration combination is identified, the combination of different pulse lengths and different duty cycles can be analyzed. For example, four different pulse lengths (100, 200, 300, and 400 ms) and four different duty cycles (50%, 20%, 10%, and 5%) can be analyzed. Again, multiple (e.g., at least ten) experiments can be performed for each set of parameters.

For the entire series of experiments, the identical skull compound can be used. To avoid bacterial growth inside the transducer, water can be replaced periodically after each series of experiments. The mounting of the skull on the acrylic plate can be fixed permanently to avoid dislocation and to improve reproducibility. To avoid air trapping, the skull can be immersed into degassed water for a period of time (e.g., 72 hour) prior to the first experiment and kept in 100% humidity for the entire time period. To improve degassing, the skull specimens can be kept in a dedicated vacuum chamber.

These series of experiments can be used to optimize the focused ultrasound parameter combinations to achieve transcranial sonothrombolysis. Because inter-individual transcranial sound field characteristics vary widely, the three most efficient combinations in a series of four more cadaveric skulls can be tested. The data obtained indicates the impact of skull bone variations on sonothrombolysis. Acoustic data can be acquired for each skull and at any point in time, due to the hydrophone measurements. Using these data, the different acoustic characteristics can be described precisely in each case. From this, the appropriate ultrasound parameter or parameter combination can be identified to achieve optimal efficacy.

Once an optimized parameter setting is identified, temperature measurements using a dedicated thermocouple can be used to monitor the temperature changes at the clot site. These measurements are performed in water, and thus, the speed of sound is different from tissue. Therefore, the thermocouple measurements are repeated once the knowledge gained is translated to an in vivo rabbit sonothrombolysis model, insonating through human cadaveric skulls. This can provide a closer estimate if thermal effects are involved applying the focused ultrasound to tissue, similar to the brain with regard to sound absorption. Various combinations of the parameters are considered to adapt parameter settings appropriately to achieve efficient sonothrombolysis without heating the surrounding tissue.

Effect of Skull Thickness

In one aspect, the power level applied for the low-power focused ultrasound can be varied based on the skull thickness of each target patient. To identify the relationship between the power level and the skull thickness, bone density and bone thickness are compared with acoustic signal absorption in a larger series of human cadaveric skulls. In addition, these three parameters are tested to determine whether they correlate with each other.

Based on the determined correlation, a coefficient can be generated to compensate for inter-individual differences in bone density and/or bone thickness to achieve comparable acoustic intensity exposure at the target site. This correlation coefficient can contribute to provide optimal US exposure for each individual stroke patient by compensating for individual bone characteristics.

Data Acquisition

Multiple volumetric Computer Tomography (CT) data sets of cadaveric skulls can be acquired to generate bone density and bone thickness maps. In addition, the transcranial sound fields of these skulls can be characterized to assess the amount of the individual signal absorption and defocusing. For example, 100 or so 3D CTdata sets of cadaveric human skulls can be acquired. Then, bone density maps can be generated based on the acquired 3D CT data sets. Also, bone thickness maps can be generated based on the 3D CT data sets. Further, transcranial sound fields of the 100 cadaveric human skulls can be assessed.

3D CT Data Acquisition

Using a CT scanner, such as the 64 slice GE Discovery HD-750 CT scanner, volume CT data sets of the skull specimens can be generated. To avoid air trapping, the skulls can be immersed into a water bath for a period or time (e.g., 72 hours) prior to the scan. In preparation for the scanning the water soaked skulls will be transferred into a housing (e.g., a 35.0×35.0×35.0 cm acrylic tank). The tank is filled with degassed, deionized water to mimic soft tissue. High resolution CT scans of the skull can be acquired with a slice thickness of 0.23 mm and zero spacing between the slices. The slices can be reconstructed using a bone filter.

Computation of Bone Density/Bone Thickness Maps

Software tools and algorithms can be implemented to reconstruct the skull bone structure. For example, the software tools from InSightec Inc. can be used. The software tools can be based on a high resolution CT scan and can result in a full reconstruction of the geometry of the skull. The different layers of the skull can be analyzed (i.e. external/internal pars compacta, medial pars spongiosa) and the local mean bone density, for example, is estimated.

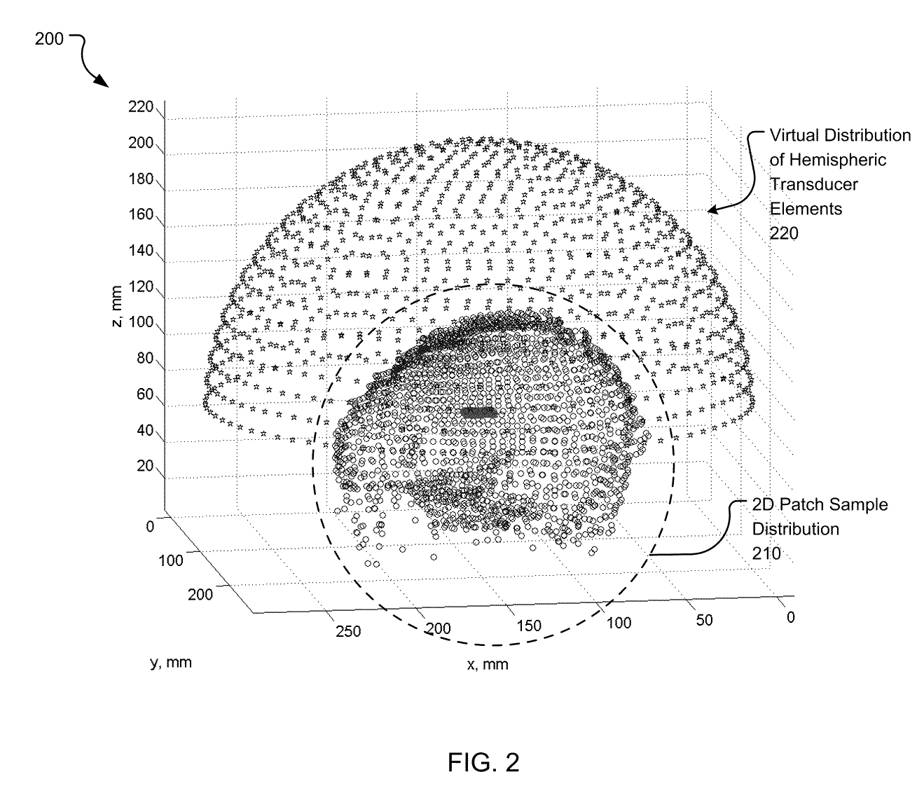

FIG. 2 a chart 200 that shows an example 3D patch sample distribution (open circles 210) on the surface of the virtual skull based on the reconstructed CT data. Black stars 220 represent virtual distribution of the hemispheric transducer elements. As shown in FIG. 2, the surface of the skull is virtually broken down to 1000 sub-areas (‘patches’) to provide a robust analysis.

In each patch, the software can be used to assess the locally unique thickness, density and geometric characteristics. For each such patch a set of samples can be taken from the CT volumetric data. The averaging of these samples can result in the following local characteristics: (1) mean bone density, (2) mean bone thickness, (3) average external cortex thickness, (4) average internal cortex thickness, and (5) average marrow thickness.

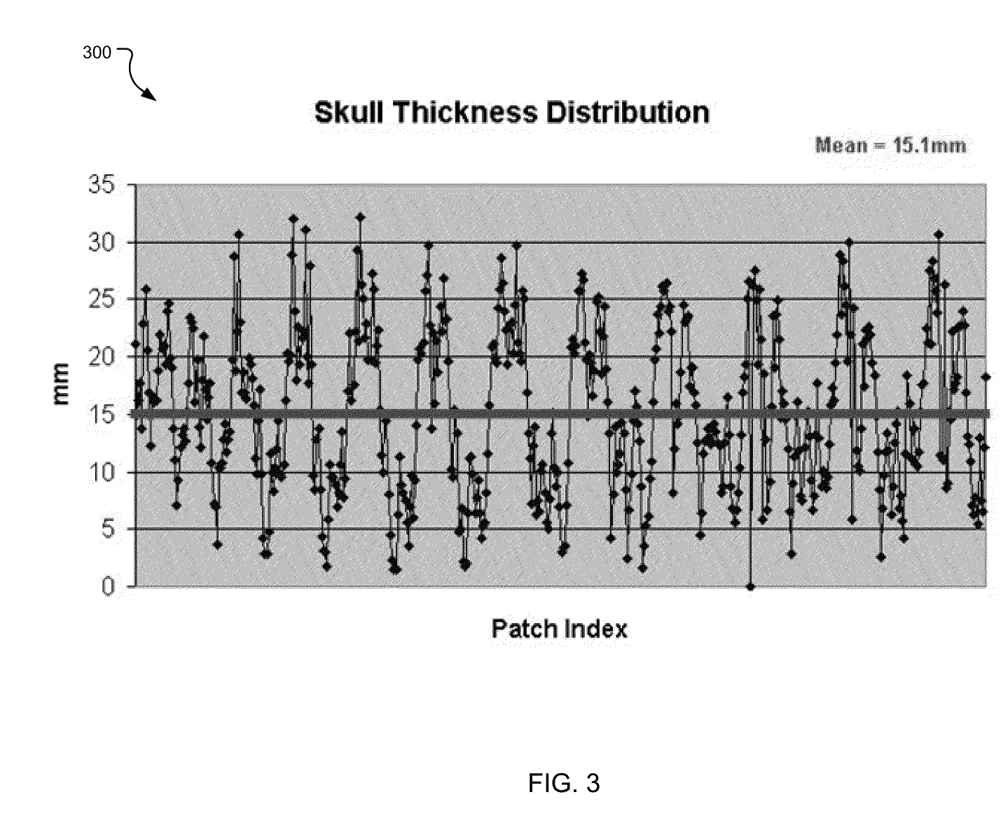

As an example, FIG. 3 is a chart 300 that shows an overall skull thickness distribution of all patches and the calculated mean thickness for the entire sample. The calculations can be done for mean bone density, different bone layers or certain skull locations (e.g., bilateral temporal bone). Skull thickness distribution for each patch is shown, resulting in a mean thickness of 15.1 mm of the entire sample, for example.

Transcranial Sound Field Characterization

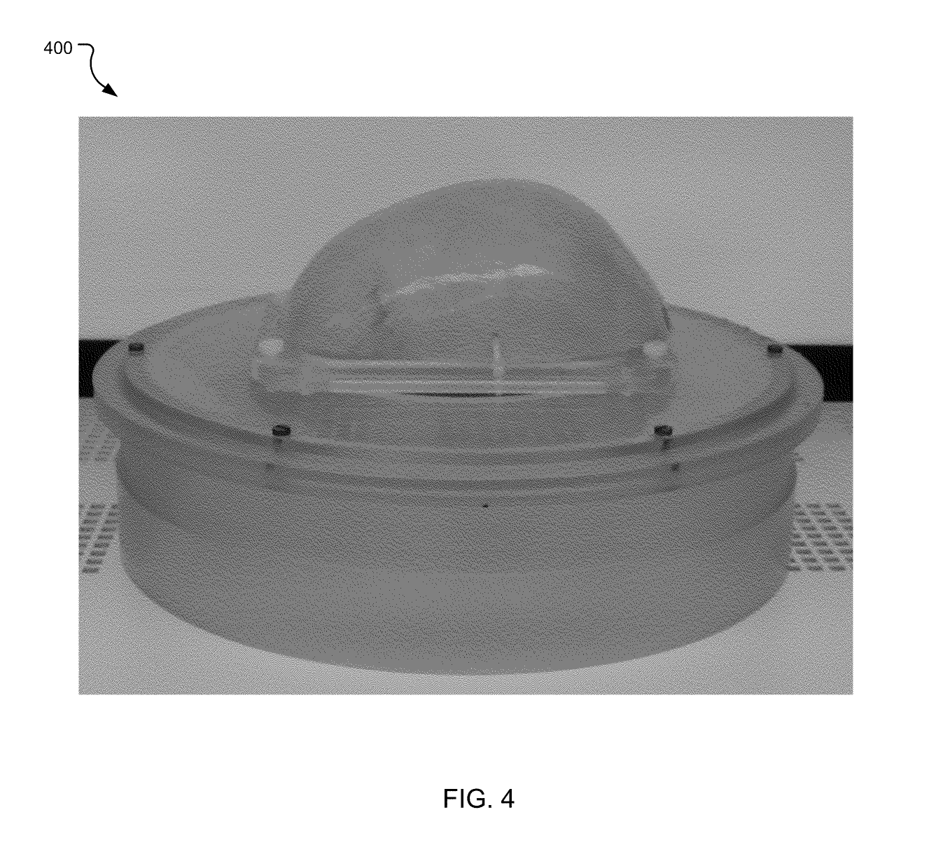

After acquisition of the CT data, the skulls can be immersed into a water bath. After 24 hours, skulls can be used for the sound field measurements. The skulls are mounted on an acrylic frame and placed upside down onto the degassed water filled hemispheric transducer. FIG. 4 is an image 400 that shows an example mounting of a skull on an acrylic frame which is fixed to a plastic cover including a center hole to provide access to the skull cavity. Once the skull is in position the entire device is placed upside down on top of the water filled hemispheric transducer.

A high intensity focused ultrasound (HiFU) hydrophone can be steered into the skull cavity towards the location of the focal spot. From this center point a two dimensional (e.g., 2.0 cm×2.0 cm) field can be scanned in XY-orientation using a stepwidth of 0.5 mm, for example. For data acquisition, a HNR series needle hydrophone (ONDA Inc., Sunnyvale/Calif., USA), designed for HiFU acoustic field mapping can be used used. The HNR series needle hydrophones are excellent sensors for laboratory use in HiFU field mapping, with pinpoint access and good spatial resolution. Due to their high sensitivity these hydrophones are commonly operated without amplification.

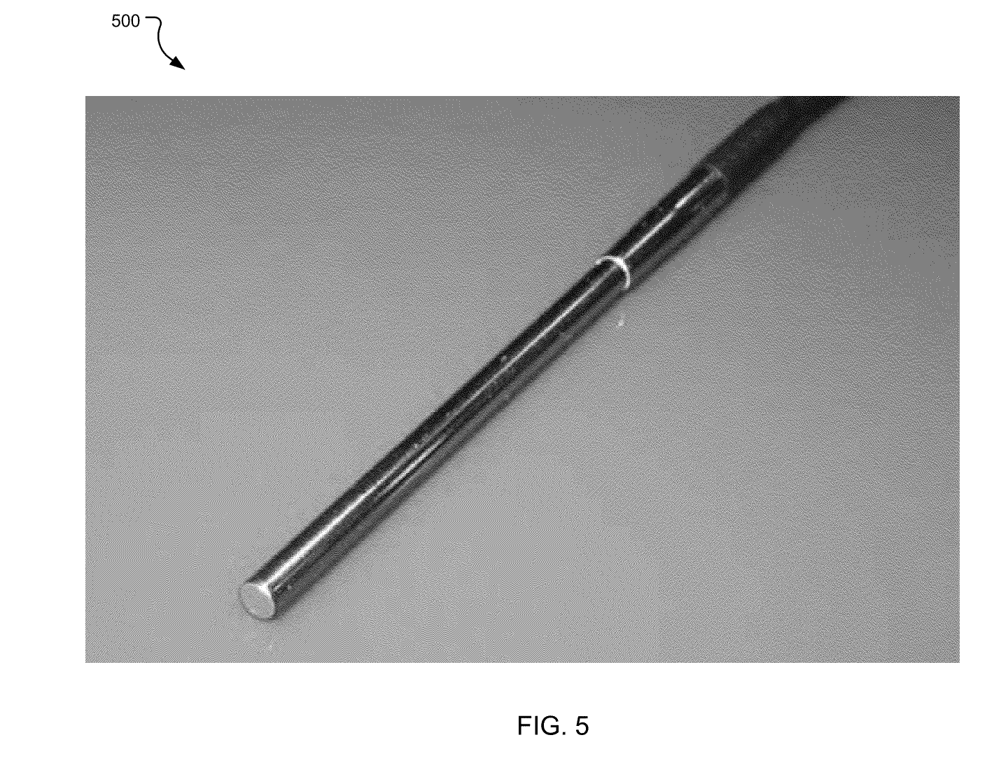

FIG. 5 is an image 500 that shows an example HiFU needle hydrophone. The hydrophone is calibrated for a minimum frequency of 250 KHz. The transmit frequency of the ExAblate™ 4000 head system is 230 KHz. The hydrophone may be recalibrated to assure that there will be no significant sensitivity loss at this lower frequency. The hydrophone shown in FIG. 5 is presented as an example only and other similarly characterized hydrophones can be implemented.

Scanning System for Measuring and Mapping Acoustic Fields in Liquids

A precision scanning system, such as Acoustic Intensity Measurement System (AIMS) from ONDA Inc. of Sunnyvale/Calif., USA can be mounted on a tank to measure and map acoustic fields in liquids. AIMS can include an associated software tool for processing acquired acoustic data. The scanning system can be suitable for medical imaging, pulsed and CW Doppler, therapeutic devices and industrial ultrasound between 0.25 and 60 MHz. The scanning system can be used to evaluate prototype transducers and excitation systems from measurements, such as: 1) Beam Dimensions; 2) Focal Zone Location and Size; 3) Pulse Amplitude, Spectrum, Center Frequency and Bandwidth; and 4) Pitch-Catch or Pulse Echo Measurement Modes. Also, the scanning system can be designed to measure, calculate and plot 1) Pulse waveform and pulse intensity integral plots; 2) Intensity: pulse average (Isppa), temporal peak(Isptp), temporal average(Ispta), pulse duration; 3) Mechanical Index; 4) In-water and derated intensity values; 5) Pressure: positive and negative peak values; 6) Acoustic power by planar integration; 7) Beam dimensions (1 and 2 dimensions); and 8) Center frequency and bandwidth. Additionally, the scanning system can be designed to compute 1) Effective Radiated Area (ERA); and 2) Beam Non-Uniformity Ratio (BNR).

Once the acoustic signals are acquired, various data acquisition and processing can be performed including waveform acquisition, different data calculations, and data output. A software tool associated with the scanning system (e.g., the AIMS software) can be used to perform the above described data processing.

Waveform acquisition can include waveform averaging using cross-correlation to remove jitter caused by vibration. Jitter can be a problem when acquiring a waveform right after moving on the z-axis, causing the membrane hydrophone to vibrate like a drum head. Additionally, acquisition of one-dimensional and two-dimensional scans can be performed. Scans can be performed on any axis or combination of axes, either translation or rotation.

Data calculations can be performed to calculate intensities (e.g., pulse average Isppa, temporal average Ispta) and pressure (e.g., peak rarefactional Pr). Also, the mechanical index (MI) and thermal indices (TIS, TIB, TIC) can be calculated. Additionally, the associated acoustic parameters such as pulse duration, beam widths, center frequency, etc. can be calculated. The calculations can include calculation of acoustic power from x-y scan data. In addition, the Effective Radiating Area (ERA) and Beam Non-uniformity Ratio (BNR) can be calculated according to FDA and IEC procedures. This includes automatic execution of the various scans required. Also, the multiple-frequency hydrophone calibration data can be calculated to obtain interpolation of hydrophone sensitivity to measured center frequency (Fc). Additionally, hydrophone calibration can be performed via planar scanning of calibrated source.

Various data can be output for storage and display to the user, for example. For example, the output files (e.g., the AIMS files) can include tab-delimited text with extensive labeling. The output files can be viewed with any text editor or can be opened with Microsoft Excel or other programs that support tab-delimited files. The format is self-explanatory. The data output can be organized, presented and printed by generating various plots and text data. The generated plots and text data can be copied to the clipboard, and then pasted into documents to prepare reports. Waveform and XY scan data can be output to a Microsoft Excel workbook for further analysis, for example. A template file can be opened by the scanning system program (e.g., AIMS program), measurement data can be copied into it, and then the copied measurement data can be written out to a new Excel file. The output worksheet in the template file can be modified to suit users' needs.

For all transcranial sound field characterizations the ultrasound ablation system 120 (e.g., the ExAblate™ 4000) can be set up with a ‘Standard’ parameter setting. The following is an exemplary standard parameter setting.

Frequency: 230 KHz

Acoustic Intensity: 300 W

Pulse Length: 100 ms

Duty Cycle: 50%

This parameter setting can be adjusted to take into account different factors, such as a) the reproducibility of the experiments, which means the parameter setting should not change between skulls and b) the acoustic intensity should be high enough to achieve reasonable (measurable) signal intensities intracranially. An acoustic intensity of 300 W is high enough to accomplish the latter even in the thickest skulls. The beamforming itself is relatively independent of the waveform parameters (i.e. pulse length, duty cycle etc). When a beam is formed, it is normally done without regard to the waveform that will be transmitted. Beamforming is like a lens on a camera, and the waveform is more like the color of light going through the lens. The beamforming is done to be as independent of the waveform as possible, just like an achromatic lens is supposed to be independent of the color of light going through it. Assuming the beamformer is properly focused, the waveform that is transmitted determines the intensity.

Baseline Sound Field Assessment without Skull

To assess and compare the intra- and inter-individual skull differences in signal absorption as well as other sound field characteristics (i.e. Effective Radiating Area) a baseline scan can be performed without a skull in place, using the identical parameter setting.

Data Analysis

The obtained CT data can be used to determine whether the bone density correlates with acoustic signal absorption. Also, a determination can be made on whether bone thickness correlates with acoustic signal absorption. In addition, a correlation between bone density and bone thickness can be assessed. Further, the combined relationship of bone density and bone thickness can be assessed for correlation with signal absorption.

Other Factors for Low Power Focused Ultrasound

Factors that impact sonothrombolysis include thrombus characteristics, such as age, fibrin-, lipid- and Calcium-content and flow mechanics (i.e. ‘water hammer’ effect, microcirculation inside the clot).

Example Skull Data

The following describes example Skull data for three skulls tested. For all three skulls, the output power for the low intensity/power ultrasound ablation system is set at 270 Watts. However, the power setting can be adjusted based on the factors described above. The skulls are different in thickness which is represented by the average clot weight loss:

Skull #1: 43%

Skull #2: 36%

Skull #3: 10%

In Skull #1 and #2 full clot liquefication is achieved at the focal spot. For Skull #3, which is significantly thicker than the other two, full clot liquefication is achieved by increasing the output power to 750 Watts.

To achieve complete liquefication of a 4 hour old clot within 30 sec, 750 Watts of maximum power can be applied. In average, the mean intensity can include the 200 Watts range.

Mechanical Ablation of Blood Clots in an Artery Supplying Blood to the Brain

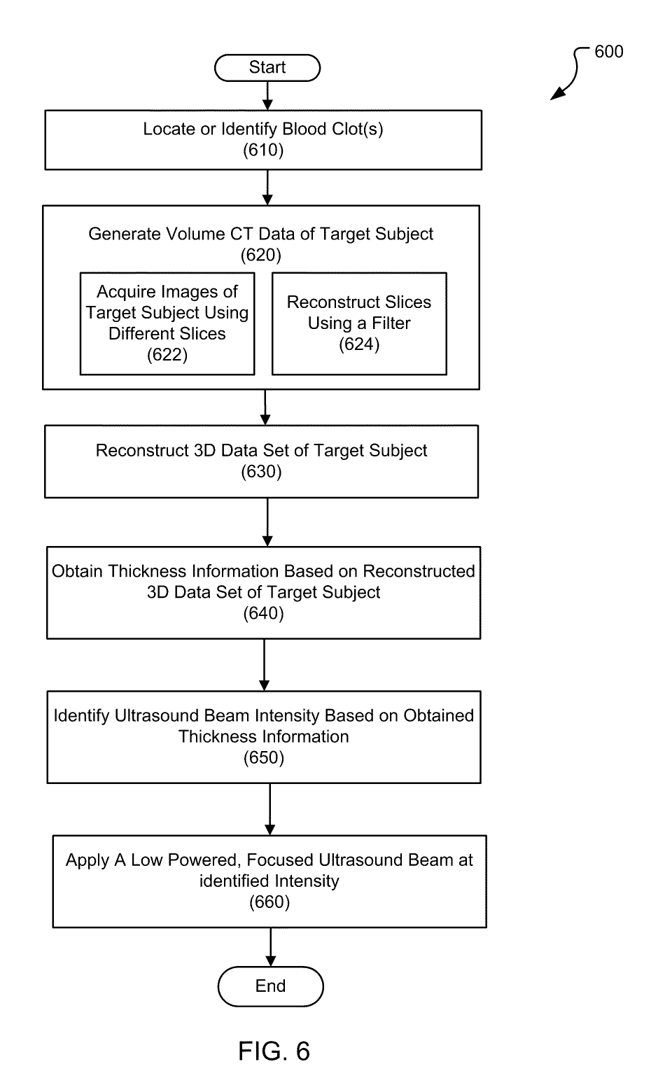

FIG. 6 shows an example process 600 for performing mechanical ablation of blood clots using focused ultrasound. A system can be used to perform CT scans of a target subject locate or identify a blood clot (610). For stroke application, the skull of the target subject can be scanned. The system can generate volume CT data of the target subject (620). In generating the volume CT data, the system can perform axial CT scans at different slices of different slice thickness (622). The system can reconstruct image slices of the target subject (624). When scanning the skull of the target subject, the system can use a bone filter to reconstruct the image slices of the skull. The system can reconstruct a 3D data set of the target subject based on the generated volume CT data (630). When scanning the skull, the generated volume CT data can be used to reconstruct a 3D data set of the skull. The system can obtain thickness information based on the reconstructed 3D data (640). For example, the system can calculate an average bone thickness of the skull based on the reconstructed 3D data. The system can identify ultrasound beam intensity based on the thickness information (650). For stroke application, the individual bone thickness value, averaged over multiple (e.g., 1,000) measurement points distributed over the entire skull, can be multiplied with a ‘bone thickness coefficient’ as described above. Based on this new calculated value, the system can identify the ultrasound intensity needed to achieve mechanically ablate the blood clots at the target site. The system can apply a low-powered, focused ultrasound beam at the identified intensity to mechanically ablate the blood clots (660).

Mechanical Ablation for Stroke Treatment

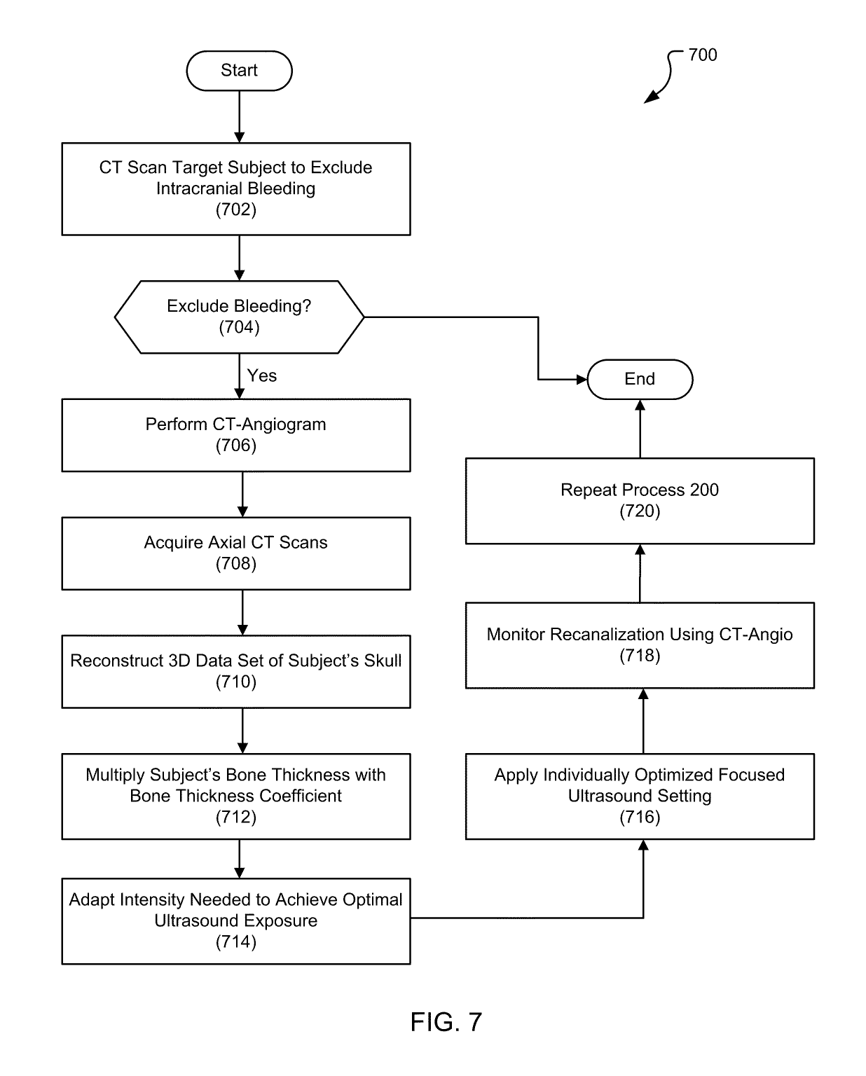

The process for performing mechanical ablation of blood clots can be implanted as a stroke treatment. FIG. 7 shows an example process for performing mechanical ablation using focused ultrasound for stroke treatment. A CT scanner is used to CT scan the target subject (702). For example, immediately after admission to the Emergency Department and neurological status assessment, the patient can be transferred to the CT scanner to exclude intracranial bleeding as a cause of the neurological deficit. Once bleeding can be excluded (704), a CT-angiogram can be performed to confirm/disconfirm an arterial vessel occlusion, causing an ischemic stroke (>80% of all strokes are ischemic) (706). In case of confirmed vessel occlusion, axial CT scans with a slice thickness of 1.0 mm, no interslice gap, is acquired right after and reconstructed using the bone filter (708). The acquired CT data is used to reconstruct a 3D data set of the patient's skull, based on which the average bone thickness is computed, using a customized software tool (710). The individual bone thickness value, averaged over 1,000 measurement points distributed over the entire skull, is multiplied with a ‘bone thickness coefficient’, as described above (712). Based on this new calculated value, the intensity needed to achieve optimal ultrasound exposure at the target site is adapted accordingly (714). The individually optimized focused ultrasound setting is applied (716) and recanalization is monitored using CT-angio consecutively (718). The procedure 200 is repeated in case reperfusion cannot be achieved immediately (720).

In some implementations, either CT or MRI can be used to show whether the patient still has brain at risk for extension of the stroke. This can be done by CT using CT perfusion and looking for mismatch between the cerebral blood volume map (core of infarct) and the cerebral blood flow or mean transit time map. MR looks for a mismatch between the diffusion abnormality (core) and the MTT map from the perfusion study. An advantage of using CT is that it is already next to the Emergency room while MRI is usually further away.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, or code) include machine instructions for a programmable processor, and may be implemented in a high-level procedural and/or object-oriented programming language, and/or in assembly/machine language. As used herein, the term “information carrier” comprises a “machine-readable medium” that includes any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal, as well as a propagated machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a WAN, and the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Only a few implementations and examples are described and other implementations, enhancements and variations can be made based on what is described and illustrated in this application.

What is claimed is:

1. A system comprising:
a computed tomography (CT) scanner to
acquire axial scans of a target subject containing at least one intravascular blood clot, and
generate volumetric CT data of the target subject based on the acquired axial scans;
a data processing device in communication with the CT scanner to
process the generated volumetric CT data, and
identify at least one parameter of an ultrasound beam to ablate the at least one intravascular blood clot based on the processed volumetric CT data; and
an ultrasound ablation device in communication with the data processing device to
generate the ultrasound beam based on the identified at least one parameter, and
apply the generated ultrasound beam with the identified at least one parameter to the target subject to ablate the at least one intravascular blood clot.

2. The system of claim 1, wherein the at least one parameter of the ultrasound beam comprises at least one of a transmit frequency, a peak intensity, a pulse length, an ultrasound duration, or a duty cycle of the ultrasound beam.

3. The system of claim 2, wherein the peak intensity comprises a level that mechanically ablates the intravascular blood clot without causing heat induced tissue damage.

4. The system of claim 1, wherein the data processing device is configured to process the generated volumetric CT data to obtain thickness or density information for a portion of the target subject using the obtained volumetric CT data.

5. The system of claim 4, wherein the data processing device is configured to identify the at least one parameter of the ultrasound beam based on the obtained thickness or density information.

6. The system of claim 5, wherein the thickness information comprises bone thickness of a skull.

7. The system of claim 6, wherein the data processing device is configured to
identify a correlation between the peak intensity of the ultrasound beam and the thickness of the skull by comparing a bone density and a bone thickness of the skull with acoustic signal absorption in the skull;
generate a coefficient to compensate for inter-individual differences in bone density or bone thickness; and
identify the at least one parameter of the ultrasound beam based on the generated coefficient.

8. A method comprising:
acquiring, at a computed tomography scanner, axial scans of a target subject containing at least one intravascular blood clot;
generating, at the computed tomography scanner, volumetric CT data of the target subject based on the acquired axial scans;
processing, at a data processing device, the generated volumetric CT data;
identify, at the data processing device, at least one parameter of an ultrasound beam to ablate the at least one intravascular blood clot based on the processed volumetric CT data;
generating, at an ultrasound ablation device, the ultrasound beam based on the identified at least one parameter; and
applying, from the ultrasound ablation device, the generated ultrasound beam with the identified at least one parameter to the target subject to ablate the at least one intravascular blood clot.

9. The method of claim 8, wherein the at least one parameter of the ultrasound beam comprises at least one of a transmit frequency, a peak intensity, a pulse length, an ultrasound duration, or a duty cycle of the ultrasound beam.

10. The method of claim 9, wherein the peak intensity comprises a level that mechanically ablates the at least one intravascular blood clot without heating tissues surrounding the intravascular blood clot.

11. The method of claim 10, comprising focusing the applied ultrasound beam to the at least one intravascular blood clot.

12. The method of claim 8, wherein processing the generated volumetric CT data comprises obtaining thickness or density information for a portion of the target subject using the obtained volumetric CT data.

13. The method of claim 12, comprising identifying the at least one parameter of the ultrasound beam based on the obtained thickness or density information.

14. The method of claim 13, wherein the thickness information comprises bone thickness of a skull.

15. The method of claim 14, comprising:
identifying a correlation between the peak intensity of the ultrasound beam and the thickness of the skull by comparing a bone density and a bone thickness of the skull with acoustic signal absorption in the skull;
generating a coefficient to compensate for inter-individual differences in bone density or bone thickness; and
identifying the at least one parameter of the ultrasound beam based on the generated coefficient.

16. A computer readable medium embodying instructions when executed by a processor cause a data processing apparatus to perform operations comprising:
acquiring axial scans of a target subject containing at least one intravascular blood clot;
generating volumetric CT data of the target subject based on the acquired axial scans;
processing the generated volumetric CT data;
identify at least one parameter of an ultrasound beam to ablate the at least one intravascular blood clot based on the processed volumetric CT data;
generating the ultrasound beam based on the identified at least one parameter; and
applying the generated ultrasound beam with the identified at least one parameter to the target subject to ablate the at least one intravascular blood clot.

17. The computer readable medium of claim 16, wherein the at least one parameter of the ultrasound beam comprises at least one of a transmit frequency, a peak intensity, a pulse length, an ultrasound duration, or a duty cycle of the ultrasound beam.

18. The computer readable medium of claim 17, wherein the peak intensity comprises a level that mechanically ablates the at least one intravascular blood clot without heating tissues surrounding the intravascular blood clot.

19. The computer readable medium of claim 18, wherein the instructions are adapted to cause a data processing apparatus to focus the applied ultrasound beam to the at least one intravascular blood clot.

20. The computer readable medium of claim 16, wherein processing the generated volumetric CT data comprises obtaining thickness or density information for a portion of the target subject using the obtained volumetric CT data.

21. The computer readable medium of claim 20, wherein the instructions are adapted to cause a data processing apparatus to identify the at least one parameter of the ultrasound beam based on the obtained thickness or density information.

22. The computer readable medium of claim 21, wherein the thickness information comprises bone thickness of a skull.

23. The computer readable medium of claim 22, wherein the instructions are adapted to cause a data processing apparatus to perform operations comprising:
identifying a correlation between the peak intensity of the ultrasound beam and the thickness of the skull by comparing a bone density and a bone thickness of the skull with acoustic signal absorption in the skull;
generating a coefficient to compensate for inter-individual differences in bone density or bone thickness; and
identifying the at least one parameter of the ultrasound beam based on the generated coefficient.
